(19) United States
(12) Patent Application Publication
(10) Pub. No.: US 2025/0250248 A1
(43) Pub. Date: Aug. 7, 2025

(54) METHOD FOR FIXATING CARBON DIOXIDE WITH EPOXIDE TO FORM A SUBSTITUTED CYCLIC CARBONATE

(71) Applicant: KING FAHD UNIVERSITY OF PETROLEUM AND MINERALS, Dhahran (SA)

(72) Inventor: Aasif HELAL, Dhahran (SA)

(73) Assignee: KING FAHD UNIVERSITY OF PETROLEUM AND MINERALS, Dhahran (SA)

(21) Appl. No.: 18/597,592

(22) Filed: Mar. 6, 2024

(30) Foreign Application Priority Data
Feb. 4, 2024 (SA) 1020240497

(51) Int. Cl. C07D 317/38; B01J 31/16

(52) U.S. Cl. CPC C07D 317/38; B01J 31/1691; B01J 2231/34; B01J 2531/48

(57) ABSTRACT

A method of fixating carbon dioxide to a substituted cyclic carbonate includes a reaction of an epoxide and carbon dioxide in the presence of a zirconium-containing metal-organic framework to form the substituted cyclic carbonate. The zirconium-containing metal-organic framework is a UiO-66-based metal-organic framework including an aminomethylbenzoic acid. The zirconium-containing metal-organic framework catalyzes the formation of the substituted cyclic carbonate in a range of 40 to 97 percent yield.

### CROSS REFERENCE TO RELATED APPLICATION

The present disclosure claims the benefit of Saudi patent application Ser. No. 10/202,40497 filed on Feb. 4, 2024, with the Saudi Authority for Intellectual Property Office, which is incorporated herein by reference in its entirety.

### STATEMENT OF ACKNOWLEDGEMENT

Support provided by the Interdisciplinary Research Center for Hydrogen and Energy Storage (IRC-HES), King Fahd University of Petroleum and Minerals, Saudi Arabia, through Project INHE2306 is gratefully acknowledged.

### BACKGROUND

### Technical Field

The present disclosure is directed to a method of fixating carbon dioxide (CO₂), more particularly a method of fixating carbon dioxide with an epoxide to form a substituted cyclic carbonate in the presence of a zirconium-containing metal-organic framework (Zr-MOF).

### Description of Related Art

The “background” description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present invention.

The widespread utilization of fossil fuels as the primary energy source for automobiles and businesses has led to a steady rise in atmospheric carbon dioxide (CO₂) levels, which has caused anomalous global climate change. CO₂ is inexpensive, abundant, and may be used as the C1 building blocks for many chemicals.

A chemical reaction process known as CO₂ fixation gives value-added carbon products. Carbon dioxide is a thermodynamically stable molecule, and the CO₂ fixation process is itself very challenging. Thus, reduction in CO₂ emissions and fixation of CO₂ has become an area of interest for research. Catalysts have been developed to overcome the high transfer energy barrier.

The deliberate incorporation of defects in a metal-organic framework (MOF) to disrupt the regular arrangements of atoms in a crystal lattice and the successive application of defect MOFs as heterogeneous catalysts is a large area of research today. The defects help in tailoring different MOF properties by imparting different functionalities such as enhanced catalytic performance. Engineered or inherent defects have helped in the catalytic activities of reactions such as Paal-Knorr reactions, Bronsted and Lewis acid-catalyzed reactions, esterification reactions, cyanosilylation, methanolysis, Knoevenagel condensation, oxidation of alkenes, and Michael Addition reaction. These defect sites have also been used to introduce catalytically active species, such as palladium and pyridine dicarboxylic acid, into MOFs or to increase the vacancies of the linker. Zirconium MOFs, especially UiO-66 (University of Oslo), having terephthalate as a linker, have attracted interest in recent years in the field of separation, adsorption, and catalysis [Vermoortele, F.; Bueken, B.; Le Bars, G.; de Voorde, B. V.; Vandichel, M.; Houthoofd, K.; Vimont, A.; Daturi, M.; Waroquier, M.; Van Speybroeck, V.; Kirschhock, C.; De Vos, D. E. J. Am. Chem. Soc. 2013, 135, 11465].

Octahedral oxoclusters, such as UiO-66, with face-centered cubic packing and hard acid-hard base combination have strong coordination bonds with the linkers. As a consequence, it has high resistance to shear stress, high stability in different organic solvents and water, and is stable in air up to 500° C. [Bai, Y.; Dou, Y.; Xie, L. H.; Rutledge, W.; Li, J. R.; Zhou, H. C. Chem. Soc. Rev. 2016, 45, 2327-2367]. The benzene ring of the terephthalate provides room for functionalizing the linker for catalysis; however, due to the high coordination number, the Zr⁴⁺ ion coordination sphere is overcrowded with carboxylate linkers. The UiO-66 has a narrow pore system for substrates to reach the internal active sites; therefore, to produce coordination vacancies for substrate binding and easily accessible active sites for catalysis, defects are incorporated. The defects make active sites more accessible without disturbing the topology, thermal stability, or chemical stability of the MOF.

Any chemical transformation involving CO₂ fixation is considered an affirmative ramification towards carbon management. Though the CO₂ activation by heterogeneous catalysts is limited, there have still been reports on the reduction of CO₂ to methanol and the synthesis of carbonates, cyclic carbonates, and synthetic gas. The coupling reactions of CO₂ are of two types: in the first type, coupling carbon dioxide with epoxides generate polycarbonates and cyclic carbonates [Darensbourg, D. J. Making Plastics from Carbon Dioxide: Salen Metal Complexes as Catalysts for the Production of Polycarbonates from Epoxides and CO₂. Chem. Rev. 2007, 107, 2388]; and in the second type, coupling carbon dioxide with an organozinc or another carbanion to form respective carboxylic acids [Boogaerts, I. I. F.; Nolan, S. P. Carboxylation of C—H Bonds Using N-Heterocyclic Carbene Gold(I) Complexes. J. Am. Chem. Soc. 2010, 132, 8858]. The formation of cyclic carbonates by CO₂ fixation with epoxides is considered to be an atom-economical chemical reaction. The cyclic carbonates are used as aprotic, polar, and nonflammable solvents in many reactions. Cyclic carbonates are also a precursor for the synthesis of polycarbonates and they act as an intermediate in many organic reactions and as electrolytes for industrial processes. Different types of homogeneous catalysts, such as quaternary ammonium salts, ionic liquids, transitional metal complexes, and organocatalysts, have been used in the aforementioned conversion, but they have drawbacks such as recyclability, separation of the catalysts and purification of the final products [Ma, R.; He, L. N.; Zhou, Y. B. An efficient and recyclable tetraoxo-coordinated zinc catalyst for the cycloaddition of epoxides with carbon dioxide at atmospheric pressure. Green Chem. 2016, 18, 226-231; Elmas, S.; Subhani, M. A.; Harrer, M.; Leitner, W.; Sundermeyer, J.; Muller, T. E. Highly active Cr(III) catalysts for the reaction of CO₂ with epoxides. Catal. Sci. Technol. 2014, 4, 1652-1657; Wilhelm, M. E.; Anthofer, M. H.; Cokoja, M.; Markovits, I. I. E.; Herrmann, W. A.; Kuhn, F. E. Cycloaddition of Carbon Dioxide and Epoxides using Pentaerythritol and Halides as Dual Catalyst System. ChemSusChem 2014, 7, 1357-1360 and Song, Q. W.; He, L. N.; Wang, J. Q.; Yasuda, H.; Sakakura, T. Catalytic fixation of CO₂ to cyclic carbonates by phosphonium chlorides immobilized on fluorous polymer. Green Chem. 2013, 15, 110-115]. Despite numerous advances in the field, efficient methods need to be developed, which may substantially reduce or eliminate the above limitations in the conversion reaction of CO₂ into cyclic carbonates.

Accordingly, it is an object of the present disclosure to provide a method for converting carbon dioxide with epoxide to form cyclic carbonates with a zirconium-containing metal-organic framework catalyst.

### SUMMARY

In an exemplary embodiment, a method of fixating carbon dioxide (CO₂) to a substituted cyclic carbonate is described. The method includes a reaction of an epoxide and CO₂ in the presence of a zirconium-containing metal-organic framework (Zr-MOF) to form the substituted cyclic carbonate. The zirconium-containing metal-organic framework is a UiO-66 (University of Oslo) based metal-organic framework (MOF). The UiO-66-based metal-organic framework includes an aminomethylbenzoic acid. The aminomethylbenzoic acid replaces a terephthalic acid in the UiO-66-based metal-organic framework.

In some embodiments, the aminomethylbenzoic acid is present in an amount of 5 to 50 mole percent of zirconium in the UiO-66-based metal-organic framework.

In some embodiments, a molar ratio of the terephthalic acid to the aminomethylbenzoic acid in the UiO-66-based metal-organic framework is from 1:0.1 to 1:5.

In some embodiments, the aminomethylbenzoic acid is 4-(aminomethyl) benzoic acid.

In some embodiments, the zirconium-containing metal-organic framework has a thermal stability of 450 to 550° C. (degrees centigrade) based on thermogravitimetric analysis.

In some embodiments, the zirconium-containing metal-organic framework is in the form of particles having a Brunauer-Emmett-Teller (BET) surface area of 600 to 1000 m²/g (square meters per gram).

In some embodiments, the zirconium-containing metal-organic framework is porous and has a pore size distribution of 1.0 to 2.0 nm (nanometers).

In some embodiments, the epoxide is at least one selected from the group consisting of propylene oxide, butylene oxide, hexylene oxide, allyl glycidyl ether, epichlorohydrin, styrene oxide, and phenyl glycidyl ether.

In some embodiments, the zirconium-containing metal-organic framework in an amount of 0.5 to 5 mole percent during the reacting based on a total number of moles of the epoxide, the carbon dioxide, and the zirconium-containing metal-organic framework.

In some embodiments, the method includes reacting at a pressure of 0.5 to 5 bar of CO₂.

In some embodiments, the method includes reacting for a time of 6 to 18 hours.

In some embodiments, the method includes reacting at a temperature of 60 to 120° C.

In some embodiments, the method includes reacting in a liquid phase.

In some embodiments, the percent yield is calculated by proton nuclear magnetic resonance spectroscopy.

In some embodiments, a molar ratio of the terephthalic acid to the aminomethylbenzoic acid in the UiO-66-based metal-organic framework is from 1:0.4 to 1:0.55, the epoxide is propylene oxide, and the percent yield of the cyclic carbonate is 40 to 50 percent with respect to an initial amount of the epoxide.

In some embodiments, a molar ratio of the terephthalic acid to the aminomethylbenzoic acid in the UiO-66-based metal-organic framework is from 1:0.6 to 1:0.8, the epoxide is propylene oxide, and the percent yield of the cyclic carbonate is 93 to 97 percent with respect to an initial amount of the epoxide.

In some embodiments, the zirconium-containing metal-organic framework is made by a process comprising: adding a zirconium salt, the terephthalic acid, and the aminomethylbenzoic acid to a solution of N,N-dimethylformamide and an acetic acid to form a reaction mixture; heating the reaction mixture in an autoclave to a temperature of 100 to 150° C. for a time of 20 to 30 hours to form a solid; washing the solid, and activating the solid under vacuum at a temperature of 80 to 120° C. for a time of 20 to 30 hours to form the zirconium-containing metal-organic framework.

In some embodiments, the percent yield of the zirconium-containing metal-organic framework is from 70 to 85 weight percent based on the total weight of the zirconium salt.

In some embodiments, the method of fixating CO₂ includes repeating the reaction in the presence of the zirconium-containing metal-organic framework at least 7 times and up to 9 times, where a yield of a final reaction is at least 80 percent of the yield of an initial reacting based on a cyclic carbonate product yield by proton nuclear magnetic resonance spectroscopy.

In some embodiments, a co-catalyst is not present during the reacting.

These and other aspects of the non-limiting embodiments of the present disclosure will become apparent to those skilled in the art upon review of the following description of specific non-limiting embodiments of the disclosure in conjunction with the accompanying drawings. The foregoing general description of the illustrative present disclosure and the following detailed descriptions thereof are merely exemplary aspects of the teachings of this disclosure and are not restrictive.

### BRIEF DESCRIPTION OF THE DRAWINGS

A more complete appreciation of this disclosure (including alternatives and/or variations thereof) and many of the attendant advantages thereof will be readily obtained as the same becomes better understood by reference to the following detailed description when considered in connection with the accompanying drawings, wherein:

FIG. 1A is a flow chart of a method of fixating carbon dioxide (CO₂) to a substituted cyclic carbonate, according to certain embodiments;

FIG. 1B is a flow chart of a method of making the zirconium-containing metal-organic framework (Zr-MOF), according to certain embodiments;

FIG. 2A shows the structures of the linkers: terephthalic acid (TPA) (201) and 4-(aminomethyl) benzoic acid (AMBA) (202), according to certain embodiments;

FIG. 2B shows the structures of UiO-66 (University of Oslo) (251) and UiO-66 with 40% defects (UiO-66-40) (252), according to certain embodiments;

FIG. 3 shows powered X-ray diffraction (XRD) patterns of UiO-66 obtained from a crystallographic information file (cif), for a stimulated form (301), pristine UiO-66 (UiO-66-0) (302), UiO-66 with 10% defects (UiO-66-10) (303), UiO-66 with 20% defects (UiO-66-20) (304), UiO-66 with 30% defects (UiO-66-30) (305), and UiO-66-40 (306), according to certain embodiments;

FIG. 4 shows partial digestion proton nuclear magnetic resonance (1H NMR) of UiO-66-10 (401), UiO-66-20 (402), UiO-66-30 (403), and UiO-66-40 (404) showing the presence of the linkers (TPA and AMBA) in different ratios, according to certain embodiments;

FIG. 5 shows infrared (IR) spectra of UiO-66-0 (501), UiO-66-10 (502), UiO-66-20 (503), UiO-66-30 (504), and UiO-66-40 (505), according to certain embodiments;

FIG. 6 shows thermal stability of UiO-66-0 (601), UiO-66-10 (602), UiO-66-20 (603), UiO-66-30 (604), and UiO-66-40 (605), according to certain embodiments;

FIG. 7 shows N₂ adsorption isotherms of UiO-66-0, UiO-66-10, UiO-66-20, UiO-66-30, and UiO-66-40, according to certain embodiments; and

FIG. 8 shows recycle tests with UiO-66-40 for the reaction of CO₂ with propylene oxide (PO) to form cyclic carbonates, according to certain embodiments.

### DETAILED DESCRIPTION

In the following description, it is understood that other embodiments may be utilized, and structural and operational changes may be made without departure from the scope of the present embodiments disclosed herein.

When describing the present disclosure, the terms used are to be construed in accordance with the following definitions, unless a context dictates otherwise. Embodiments of the present disclosure will now be described more fully hereinafter with reference to the accompanying drawings wherever applicable in which some, but not all, embodiments of the disclosure are shown. Reference will now be made to specific embodiments or features, examples of which are illustrated in the accompanying drawings. In the drawings, whenever possible, corresponding or similar reference numerals will be used to designate identical or corresponding parts throughout the several views. Moreover, references to various elements described herein are made collectively or individually when there may be more than one element of the same type; however, such references are merely exemplary in nature. In may be noted that any reference to elements in the singular may also be constructed to relate to the plural and vice-versa without limiting the scope of the disclosure to the exact number or type of such elements unless set forth explicitly in the appended claims. Further, as used herein, the use of the singular includes plural and the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

The use of the terms “include,” “includes,” “including,” “have,” “has,” or “having” should be generally understood as open-ended and non-limiting unless specifically stated otherwise.

As used herein, the term “alkyl,” unless otherwise specified, refers to a straight, branched, or cyclic, saturated aliphatic fragment having 1 to 26 carbon atoms, (e.g., C1, C2, C3, C4, C5, C6, C7, C8, C9, C10, C11, etc.) and specifically includes, but is not limited to, methyl, ethyl, propyl, isopropyl, butyl, isobutyl, t-butyl, pentyl, isopentyl, neopentyl, hexyl, isohexyl, 3-methylpentyl, 2,2-dimethylbutyl, 2,3-dimethylbutyl, 2-ethylhexyl, heptyl, octyl, nonyl, 3,7-dimethyloctyl, decyl, undecyl, dodecyl, tridecyl, tetradecyl, pentadecyl, hexadecyl, heptadecyl, octadecyl, nonadecyl, eicosyl, guerbet-type alkyl groups (e.g., 2-methylpentyl, 2-ethylhexyl, 2-propylheptyl, 2-butyloctyl, 2-pentylnonyl, 2-hexyldecyl, 2-heptylundecyl, 2-octyldodecyl, 2-nonyltridecyl, 2-decyltetradecyl, and 2-undecylpentadecyl), as well as cyclic alkyl groups (cycloalkyls) such as cyclopropyl, cyclobutyl, cyclopentyl, cyclohexyl, norbornyl, and adamantyl.

As used herein, the term “epoxide,” unless otherwise specified, refers to a cyclic ether with a three-atom ring.

As used herein, the term “substituted” refers to at least one hydrogen atom that is replaced with a non-hydrogen group, (i.e., a functional group) provided that normal valencies are maintained and that the substitution results in a stable compound. Examples of functional groups may include, but are not limited to, alkyl, alkenyl, alkynyl, phenyl, halo, hydroxyl, ketyl, aldehyde, haloformyl, carbonate ester, carboxylate, carboxyl, carboalkoxy, hydroperoxyl, peroxy, ether, ester, hemiacetal, hemiketal, acetal, ketal, orthoester, methylenedioxy, orthocarbonate ester, carboxylic anhydride, carboxamide, amidine, primary amine, secondary amine, tertiary amine, hydrazone, primary amine, secondary amine, primary aldimine, secondary aldimine, imide, azide, diimide, cyanate, isocyanate, nitrate, nitrile, isonitrile, nitrosooxy, nitro, nitroso, oxime, pyridyl, carbamate, sulfhydryl, sulfide, disulfide, sulfinyl, sulfonyl, sulfino, sulfo, alkoxysulfonyl, thiocyanate, isothiocyanate, carbonothioyl, thial, thiolester, thionoester, carbiodithio, phosphino, phosphono, phosphate, borono, boronate, borino, borinate, silyl ether, combinations thereof, and the like.

Throughout the specification and the appended claims, a given chemical formula or name shall encompass all isomers (stereo and optical isomers and racemates) thereof, where such isomers exist. Unless otherwise indicated, all chiral (enantiomeric and diastereomeric) and racemic forms are within the scope of the disclosure. It should be understood that all conformers, rotamers, or conformational isomer forms, insofar as they may exist, are included within the present disclosure.

As used herein, “metal-organic frameworks” or MOFs are compounds having a lattice structure made from (i) a cluster of metal ions as vertices (“cornerstones”) (“secondary building units” or SBUs) which are metal-based inorganic groups, for example, metal oxides and/or hydroxides, linked together by (ii) organic linkers. The linkers are usually at least bidentate ligands that coordinate with the metal-based inorganic groups via functional groups, such as carboxylates and/or amines. MOFs are considered coordination polymers made up of (i) metal ion clusters and (ii) linker building blocks. The metal ion clusters are coordinated to the linker building blocks to form one-, two-, or three-dimensional structures. A metal-organic framework is a porous extended structure made from the metal ion clusters and the linker building blocks. The porous extended structure is a structure whose sub-units occur in a constant ratio and are arranged in a repeating pattern.

The present disclosure provides a zirconium metal-organic framework (Zr-MOF) that catalyzes carbon dioxide (CO₂) fixation. Alkylamine-modified defects were introduced in UiO-66 (University of Oslo) Metal-Organic Framework (MOF), and the resulting Zr-MOFs were evaluated for their potential to convert a wide range of epoxides, using CO₂, to give cyclic carbonates. The results indicate that the UiO-66 MOF with a defect of 40% based on an overall amount of linker is particularly effective in the fixation of CO₂ with epoxides to synthesize cyclic carbonates in good yield. The Zr-MOF of the present disclosure is recyclable and can be re-used for multiple cycles (e.g., eleven consecutive cycles) without significantly compromising the yield of the cyclic carbonates.

The MOF of the present disclosure is preferably based on zirconium ions (made from zirconium ion clusters), referred to herein as a zirconium metal-organic framework (Zr-MOF). The Zr-MOF herein is intended to cover any MOF that contains predominantly zirconium ions with respect to the total metal ion content. The Zr-MOF of the present disclosure preferably includes greater than 50 wt. % of zirconium ions, preferably greater than 60 wt. % of zirconium ions, preferably greater than 70 wt. % of zirconium ions, preferably greater than 80 wt. % of zirconium ions, preferably greater than 85 wt. % of zirconium ions, preferably greater than 90 wt. % of zirconium ions, preferably greater than 95 wt. % of zirconium ions, more preferably greater than 99 wt. % of zirconium ions, and yet more preferably 100 wt. % of zirconium ions, based on the total weight of metal ions present in the MOF. If additional metal ions are present (other than zirconium ions), these may be present in an amount of less than 50 wt. %, preferably less than 40wt. %, preferably less than 30 wt. %, preferably less than 20 wt. %, preferably less than 15 wt. %, preferably less than 10 wt. %, more preferably less than 5 wt. %, and yet more preferably less than 1 wt. %, based on the total weight of metal ions. Additional metal ions may include, but are not limited to, ions of lithium, beryllium, sodium, magnesium, aluminum, potassium, calcium, scandium, titanium, vanadium, chromium, manganese, iron, cobalt, nickel, copper, zinc, molybdenum, ruthenium, cadmium, hafnium, tungsten, iridium, and cerium.

The Zr-MOFs include zirconium ion clusters (cornerstones), which are zirconium inorganic groups, typically zirconium ions connected by bridging oxygen groups, bridging hydroxide groups, or both. These zirconium ion clusters are further coordinated to at least one linker. In some embodiments, the zirconium ion clusters may be further connected to non-bridging modulator species, complexing reagents, ligands, and/or solvent molecules.

In some embodiments, the zirconium ion clusters (cornerstones), the Zr-MOFs of the present disclosure are formed from at least one linker, which may be bidentate, tridentate, or tetradentate, and which links together adjacent zirconium ion clusters to form a coordinated network. In some embodiments, the at least one linker is a terephthalic acid. In a preferred embodiment, the at least one linker is terephthalic acid. In preferred embodiments, the zirconium-containing metal-organic framework is a UiO-66-based metal-organic framework.

The UiO-66-based metal-organic framework includes an aminomethylbenzoic acid. In a preferred embodiment, the aminomethylbenzoic acid is 4-(aminomethyl) benzoic acid. In some embodiments, the aminomethylbenzoic acid is present in an amount of 5 to 50 mole percent, preferably 10 to 45 mole percent, preferably 15 to 40 mole percent, preferably 20 to 35 mole percent, or preferably 25 to 30 mole percent of zirconium in the UiO-66-based metal-organic framework. During the reaction, the aminomethylbenzoic acid replaces, at least partially, terephthalic acid in the UiO-66-based metal-organic framework. In some embodiments, the molar ratio of the terephthalic acid to the aminomethylbenzoic acid in the UiO-66-based metal-organic framework is from 1:0.1 to 1:5, preferably 1:0.2 to 1:4, preferably 1:0.3 to 1:3, preferably 1:0.4 to 1:2, and preferably 1:0.5 to 1:1.

The Zr-MOF of the present disclosure has thermal stability of 450 to 550° C., preferably 460 to 540° C., preferably 470 to 530° C., preferably 480 to 520° C., preferably 490 to 510° C., and preferably about 500° C. as studied by thermogravimetric analysis (TGA).

The Zr-MOF of the present disclosure has a Brunauer-Emmett-Teller (BET) surface area of 600 to 1000 m²/g, preferably 610 to 990 m²/g, preferably 650 to 950 m²/g, preferably 700 to 900 m²/g, or preferably 750 to 850 m²/g.

The Zr-MOF of the present disclosure has a pore size of 1.0-2.0 nm (nanometers), preferably 1.1-1.9 nm, preferably 1.2-1.8 nm, preferably 1.3-1.7 nm, or preferably 1.4-1.6 nm. As used herein, “pore size” is the average distribution of diameters of pores or of vacancies in the Zr-MOF. The vacancies in the Zr-MOF may trap molecules, such as the carbon dioxide and the epoxide and allow them to react to form the cyclic carbonate product.

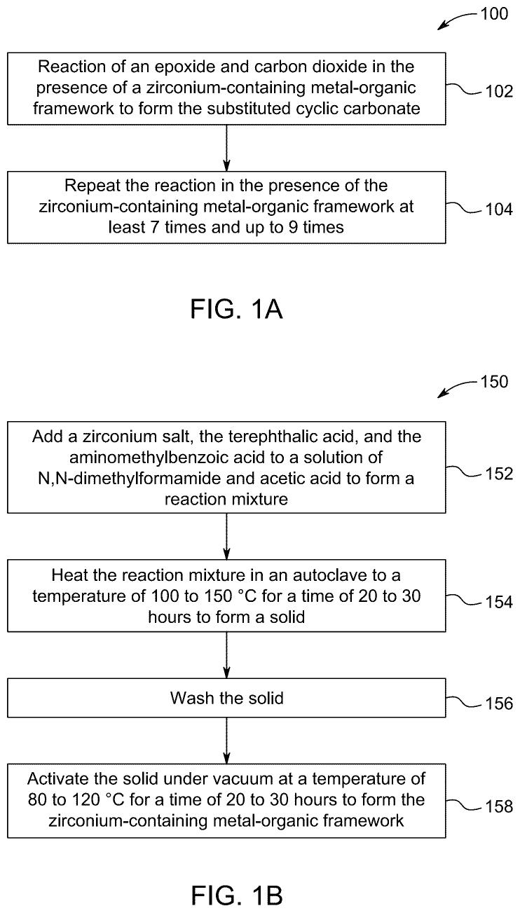

FIG. 1A illustrates a schematic flow diagram of the method 100 for fixating carbon dioxide to a substituted cyclic carbonate. The order in which the method 100 is described is not intended to be construed as a limitation, and any number of the described method steps may be combined in any order to implement the method 100. Additionally, individual steps may be removed or skipped from the method 100 without departing from the spirit and scope of the present disclosure.

At step 102, the method 100 includes reacting an epoxide and carbon dioxide in the presence of the zirconium-containing metal-organic framework (Zr-MOF) to form the substituted cyclic carbonate. The epoxide is at least one of propylene oxide, butylene oxide, hexylene oxide, allyl glycidyl ether, epichlorohydrin, styrene oxide, and phenyl glycidyl ether. In some embodiments, the epoxide may be optionally substituted. In some embodiments, the epoxide may be any epoxide known in the art. In a preferred embodiment, the epoxide is propylene oxide. The epoxide reacts with CO₂ in the presence of the Zr-MOF, which catalyzes the formation of the substituted cyclic carbonate. The reaction occurs with the zirconium-containing metal-organic framework in an amount of 0.5 to 5 mole percent, preferably 1 to 4 mole percent, preferably 1.5 to 3 mole percent, and more preferably about 2 mole percent based on the total number of moles of the reaction, including moles of CO², moles of epoxide, and moles of the catalyst.

In some embodiments, the Zr-MOF catalyzes the formation of the substituted cyclic carbonate at a pressure of 0.5 to 5 bar, preferably 1 to 3 bar, preferably about 1 bar of carbon dioxide. In some embodiments, the Zr-MOF catalyzes the formation of the substituted cyclic carbonate in a time of 6 to 18 hours, preferably 7 to 17 hours, preferably 8 to 16 hours, preferably 9 to 15 hours, preferably 10 to 14 hours, preferably 11 to 13 hours, and more preferably about 12 hours. In some embodiments, the Zr-MOF catalyzes the formation of the substituted cyclic carbonate at a temperature of 60 to 120° C., preferably 70 to 110° C., preferably 80 to 100° C., and more preferably about 85° C. In a preferred embodiment, the catalysis reaction occurs at reaction conditions of 1 bar of CO₂ at 85° C. for 12 hours. In some embodiments, the reacting occurs in a liquid phase. In other embodiments, the reacting occurs in a gas phase.

In a preferred embodiment, the reacting occurs without a co-catalyst. In some other embodiments, the reaction may be carried out in the presence of a co-catalyst. The co-catalyst may be one or more selected from zeolites, metal oxides, functional polymers, tetrabutylammonium iodide (TBAI), tetrabutylammonium bromide (TBAB), and tetrabutylammonium fluoride (TBAF), or the like.

The percentage yield of the substituted cyclic carbonate catalyzed by the catalyst of the present disclosure is in a range of 40 to 97 percent yield, preferably 50 to 96 percent yield, preferably 60 to 95 percent yield, preferably 70 to 94 percent yield, preferably 80 to 93 percent yield, and more preferably 85 to 92 percent yield. The percent yield is weight percent calculated from an amount of an isolated cyclic carbonate product with respect to an initial amount of the epoxide. In some embodiments, the percent yield is calculated by ¹H NMR spectra.

In some embodiments, a molar ratio of the terephthalic acid to the aminomethylbenzoic acid in the UiO-66-based metal-organic framework is from 1:0.4 to 1:0.55, the epoxide is propylene oxide, and the percent yield of the cyclic carbonate is 40 to 50 percent by weight with respect to an initial amount of the epoxide. In another embodiment, a molar ratio of the terephthalic acid to the aminomethylbenzoic acid in the UiO-66-based metal-organic framework is from 1:0.6 to 1:0.8, the epoxide is propylene oxide, and the percent yield of the cyclic carbonate is 93 to 97 percent by weight with respect to an initial amount of the epoxide.

At step 104, the method 100 includes repeating the reaction in the presence of the zirconium-containing metal-organic framework at least 7 times and up to 9 times. This suggests that the catalyst has potential for re-usability. The percentage yield of the final product, i.e., the substituted cyclic carbonate, was found to be greater than 70%, preferably greater than 72%, preferably greater than 75%, preferably greater than 78%, and preferably greater than 80% of an initial reaction, after re-using the zirconium-containing metal-organic framework for 7 to 9 times. The percent yield as calculated by proton nuclear magnetic resonance spectroscopy. In some embodiments, the percentage yield of the substituted cyclic carbonate was found to be in the range of 40 to 60%, preferably 45 to 55%, or preferably 47 to 52% of the initial yield after re-using the Zr-MOF 10 to 12 times. The percent yield is weight percent calculated by proton nuclear magnetic resonance spectroscopy.

Referring to FIG. 1B, a schematic flow diagram of a method 150 for making the zirconium-containing metal-organic framework (Zr-MOF) is illustrated. The order in which the method 150 is described is not intended to be construed as a limitation, and any number of the described method steps may be combined in any order to implement the method 150. Additionally, individual steps may be removed or skipped from the method 150 without departing from the spirit and scope of the present disclosure.

At step 152, the method 150 includes adding a zirconium salt, the terephthalic acid (TPA), and the aminomethylbenzoic acid (AMBA) to a solution of polar solvent such as N,N-dimethylformamide (DMF) and acetic acid to form a reaction mixture. In an embodiment, the zirconium salt is zirconium (IV) salt. Suitable examples of zirconium (IV) compounds or salts may include, but are not limited to, zirconium (IV) chloride, zirconium (IV) bromide, zirconium (IV) acetylacetonate, zirconium (IV) fluoride, zirconium (IV) hydroxide, zirconium (IV) acetate hydroxide, and zirconium (IV) trifluoroacetylacetonate, preferably zirconium (IV) chloride. In a preferred embodiment, the zirconium (IV) salt is zirconium (IV) chloride. The concentration of the zirconium salt in the reaction mixture may range from 0.01 to 0.06 M, preferably 0.02 to 0.05 M, preferably 0.03 to 0.045 M, or preferably about 0.04 M. A mixture of TPA and AMBA form the linker. In some embodiments, a molar ratio of the zirconium salt to the linker in the reaction mixture is from 1:2 to 2:1, preferably 1:1.9 to 1:1.1, preferably 1:1.8 to 1:1.2, preferably 1:1.7 to 1:1.3, preferably 1:1.6 to 1:1.4, or preferably about 1:1.5. In a preferred embodiment, the molar ratio of ZrCl₄: TPA+AMBA in the mixed solvent is 1:1.1. The zirconium salt, the linker, and the solution may be mixed by any suitable technique for any period of time until complete dissolution is achieved.

Although the preferred solvent is a solution of DMF and acetic acid, optionally, other polar aprotic solvents may be used as well, alone or in combination with the DMF. Suitable examples of the polar aprotic solvents include, but are not limited to, N-methyl-2-pyrrolidone, dimethyl sulfoxide, acetone, acetonitrile, dimethylacetamide, 1,3-dimethyl-2-imidazolidinone, the like, and/or combinations thereof.

At step 154, the method 150 includes heating the reaction mixture in an autoclave to a temperature of 100 to 150° C. for a time of 20 to 30 hours to form a solid. The heating may be carried out using heating appliances such as hot plates, heating mantles, ovens, microwaves, autoclaves, tapes, oil baths, salt baths, sand baths, air baths, hot-tube furnaces, and hot-air guns. In some embodiments, the heating is carried out in an autoclave. In some embodiments, the heating may be carried out for 20 to 30 hours, preferably 22 to 28 hours, preferably 24 to 26 hours, and more preferably about 24 hours at a temperature range of 100 to 150° C., preferably 105 to 145° C., preferably 110 to 140° C., preferably 115 to 135° C., and preferably 120 to 130° C. In a preferred embodiment, the zirconium salt, the linker, and the mixed solvent are heated for 24 hours at 120° C. to form the solid. Under such coordination conditions, the Zr-MOF of the present disclosure may be formed by self-assembly of the building blocks (i.e., zirconium ion clusters and the linker and may precipitate from solution due to insolubility in the reaction environment).

At step 156, the method 150 includes washing the solid. The solid, including the Zr-MOF, may be separated from the reaction mixture using any known solid-liquid separation technique (e.g., filtration, decantation, centrifugation, etc.) and optionally washed with a polar aprotic solvent (e.g., DMF). In preferred embodiments, the collected Zr-MOF is washed with DMF, preferably washed with DMF at least 2 times, preferably at least 3 times, with centrifugation at 5,000 to 15,000 rpm, preferably 7,000 to 12, 000 rpm, and preferably at 10,000 rpm following each washing iteration. The centrifugation is preferably done for 5 to 30 minutes, preferably 10 to 20 minutes, and preferably about 15 minutes. After washing with the polar aprotic solvent, the Zr-MOF may optionally be washed with an alcohol (e.g., methanol) at least 2 times per day, preferably at least 3 times per day. The washing may be performed for 1 to 5 days, preferably for 2 to 4 consecutive days, preferably for 3 consecutive days, and most preferably washed with methanol 3 times per day for 3 consecutive days.

At step 158, the method 150 includes activating the solid under vacuum at a temperature of 80 to 120° C. for a time of 20 to 30 hours to form the Zr-MOF. It is preferred to activate the Zr-MOF under vacuum and at elevated temperatures, for example, at 70 to 130° C., preferably 80 to 120° C., preferably 90 to 110° C., and more preferably about 100° C., for 20 to 30 hours, preferably 22 to 28 hours, preferably 24 to 26 hours, and more preferably about 24 hours, to remove any solvent molecules before use. In some embodiments, the percent yield of the Zr-MOF obtained by the method of the present disclosure is from 70 to 85 percent, preferably 72 to 83 percent, preferably 75 to 80 percent based on the zirconium salt.

### EXAMPLES

The following examples demonstrate a method of fixating carbon dioxide to a substituted cyclic carbonate, as described herein. The examples are provided solely for illustration and are not to be construed as limitations of the present disclosure, as many variations thereof are possible without departing from the spirit and scope of the present disclosure.

### Example 1: Materials

Terephthalic acid (TPA; 98% purity), zirconium tetrachloride (ZrCl₄; 99.99% purity), 4-(aminomethyl) benzoic acid (AMBA; 97% purity), methanol (MeOH; 99.9% purity), N,N-dimethylformamide (DMF; 99.8% purity), dichloromethane (DCM; 99.8% extra dry grade), propylene oxide (PO; 99% purity), with all the other epoxides purchased from Sigma Aldrich Corporation. Nuclear Magnetic Resonance (NMR) solvents: dimethyl sulfoxide-d6 (DMSO-d₆; 99.9% purity) were purchased from Cambridge Isotope. All chemicals were used without further purification. Water was double distilled and filtered through a Millipore membrane.

### Example 2: Instrumentation

Proton (¹H) and carbon-13 (¹³C) nuclear magnetic resonance (NMR) spectra were recorded on a Bruker AM-400 (manufactured by Bruker, United States) using tetramethylsilane (TMS) as the internal standard. Powder X-ray diffraction (PXRD) patterns of the samples were recorded using a Rigaku MiniFlex diffractometer (manufactured by Rigaku, Japan) equipped with Cu-Kα radiation. The data were acquired over the 2θ range of 5° and 40°. Fourier-transform infrared (FTIR) spectra were obtained using a Nicolet 6700 Thermo Scientific instrument (manufactured by Thermo Fischer Scientific, United States) in the range of 400-4000 cm⁻¹ (reciprocal centimeters), using KBr (potassium bromide) plates. Thermogravimetric analysis (TGA) was conducted using a TA Q500 with the sample in a platinum pan under airflow. The Brunauer-Emmett-Teller (BET) surface area of the metal-organic framework (MOF) and its composites were calculated by using the Micromeritics ASAP 2020 instrument (manufactured by Micrometrics, United States). A liquid nitrogen bath was used for the measurements at 77 Kelvin (K).

### Example 3: Synthesis of MOFs

The MOFs were prepared solvothermally with a slight modification of the reported method [Cavka, J. H.; Jakobsen, S.; Olsbye, U.; Guillou, N.; Lamberti, C.; Bordiga, S.; Lillerud, K. P. J. Am. Chem. Soc., 2008, 130, 13850, incorporated herein by reference in its entirety]. The synthesis was carried out with a molar ratio of ZrCl₄: TPA: AMBA (1:1-x:x) in a mixed solvent of DMF (40 mL (milliliters)) and acetic acid (2.8 mL). The reaction mixtures were mixed according to Table 1 to maintain the ratio of the linker-modified defects and transferred to teflon-lined autoclaves and heated at 120° C. (degrees centigrade) for 24 hours. The resulting solid products obtained were cooled, washed three times with DMF (5 mL) using a centrifuge (10,000 rpm for 15 minutes), and then sequentially immersed in methanol (5 mL three times per day) for three 24-hour periods. Finally, each product was activated by removing the solvent under a vacuum for 24 hours at 100° C. The obtained MOFs are denoted as UiO-66-x, where x is the percentage of AMBA in the applied linker (which is composed of both TPA and AMBA).

TABLE 1
Molar compositions of major precursors for
the synthesis of MOFs from TPA and AMBA

| | ZrCl₄(mmole)/ | TPA(mmole)/ | AMBA(mmole)/ | Yield |
|---|---|---|---|---|
| MOF^{a} | Wt. (mg) | Wt. (mg) | Wt. (mg) | (%)^{b} |
| UiO-66-0 | 1/233 | 1/167 | 0 | 80 |
| UiO-66-10 | 1/233 | 0.9/150 | 0.16/24 | 80 |
| UiO-66-20 | 1/233 | 0.8/134 | 0.27/41 | 75 |
| UiO-66-30 | 1/233 | 0.7/117 | 0.37/56 | 74 |
| UiO-66-40 | 1/233 | 0.6/100 | 0.45/68 | 72 |

^{a}Synthesis temperature: 120° C., time: 24 hours;
^{b}Based on Zr

### Example 4: Procedure for CO₂ Cycloaddition

Completely dried UiO-66-40 material (2 mmol (millimole) of Zr), epoxide (6.0 mmol) was added to a 50 mL round bottom flask attached to a balloon filled with CO₂ and heated at 85° C. for 12 hours. After completion, the reaction mixture was cooled. The reaction mixture was then diluted with methanol and centrifuged to separate the catalyst. The organic layer was concentrated, passed through a short silica column, and eluted with an ethyl acetate/hexane mixture. The pure form of the respective cyclic carbonates was dried and analyzed by ¹H NMR and ¹³C NMR.

### Example 5: Reusability Test

To reuse the catalysts, including UiO-66-40, after completion of the reaction, the reaction mixture was diluted with MeOH and the catalysts were recovered by simple centrifugation (5000 rpm). Recovered UiO-66-40 was repeatedly washed with 10 mL dichloromethane aliquots (to remove remaining propylene oxide and product) three times, washed three times with 10 mL chloroform aliquots, and then dried under vacuum overnight.

### Results and Discussion

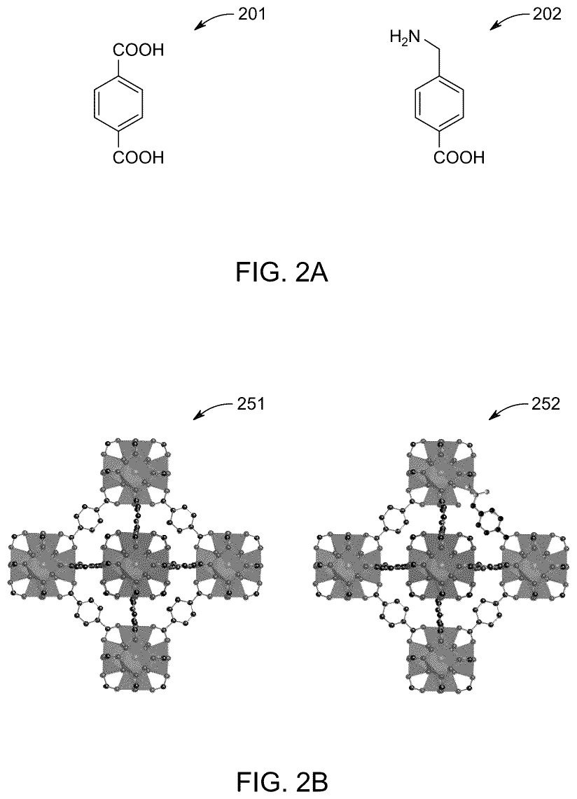

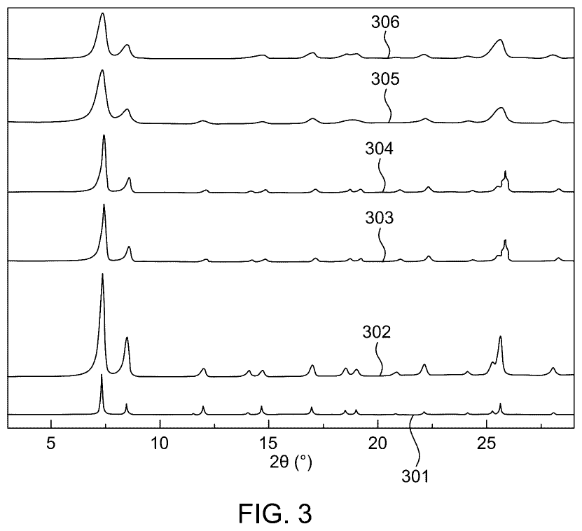

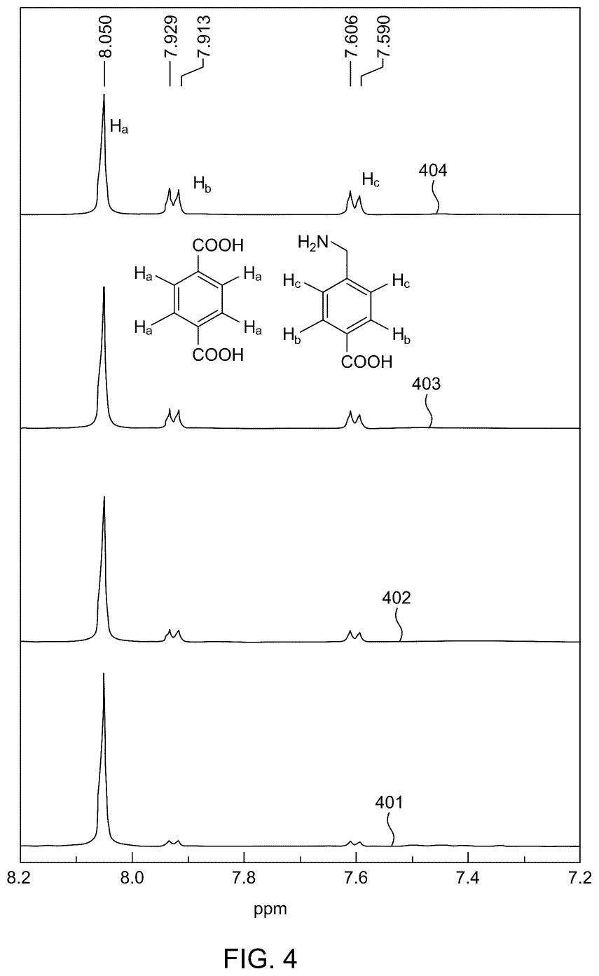

Modified linker defects in UiO-66 were introduced to increase vacant coordination sites of zirconium so that the zirconium can act as a Lewis acid and functionalize defect sites of the linker with alkyl amines to enhance the CO₂ uptake in the synthesis of cyclic carbonates from epoxides. UiO-66 with varying degrees of linker-modified defects was prepared using ZrCl₄ as the metal precursor. FIG. 2A shows the structures of the organic linkers terephthalic acid (TPA) (201) and 4-(aminomethyl) benzoic acid (AMBA) (202). Different molar ratios of the organic linkers compositions to form the various MOFs are given in Table 2. FIG. 3 shows the obtained powdered XRD patterns of the MOFs with various ratios of the TPA and AMBA. Each of the MOFs (pristine UiO-66 (302), UiO-66-10 (303), UiO-66-20 (304), UiO-66-30 (305), UiO-66-40 (306)) were found to have similar crystalline nature as the MOFs' simulated form (301). This indicates that the topology of UiO-66 remains intact until the introduction of 40% of the defects (AMBA). Beyond that, further introduction of the defects destroys the characteristic topology of UiO-66. All the activated MOFs were digested in an alkaline solution of sodium hydroxide (NaOH) and dissolved in DMSO-d₆ for ¹H NMR analysis. The digested NMR of the samples with defects (UiO-66-10 (401), UiO-66-20 (402), UiO-66-30 (403), and UiO-66-40 (404)) shows the presence of both the linkers TPA and AMBA), as can be observed in FIG. 4. The protons of each linker were assigned based on their chemical shifts and inherent splitting patterns. A designation of Hₐ was to protons of the benzene ring of TPA. A designation of H_{b} was given to protons bonded to carbons in the benzene ring one carbon from the carbon bonded to the carboxylic acid of AMBA. A designation of H_{c} was given to protons bonded to carbons in the benzene ring one carbon from the carbon bonded to the methylamine of AMBA. The relative ratio of each linker incorporated in the framework was quantitatively investigated by integrating their ¹H NMR peaks (Table 2).

TABLE 2
Input and output ratio of the linkers
obtained from the digestion NMR

| | Ratio of linkers | Ratio of linkers |
|---|---|---|
| | TPA:AMBA | TPA:AMBA output |
| MOF | input in mmole. | from NMR digestion. |
| UiO-66-10 | 1:0.18 | 1:0.11 |
| UiO-66-20 | 1:0.34 | 1:0.25 |
| UiO-66-30 | 1:0.53 | 1:0.44 |
| UiO-66-40 | 1:0.75 | 1:0.64 |

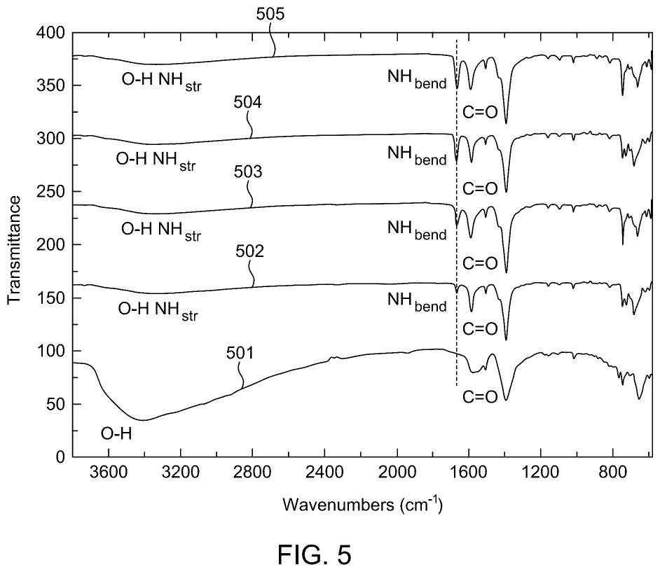

FIG. 5 shows the IR spectrum of the pristine UiO-66 (501) and UiO-66 with varying amounts of defects, namely, UiO-66-10 (502), UiO-66-20 (503), UiO-66-30 (504), and UiO-66-40 (505). The spectra indicate that with the increase in the amount of defects, a new peak at 1668 cm⁻¹ is slowly formed, which corresponds to the NH-bending frequency of the alkylamine. From FIG. 2B, it is seen that the defects were introduced and they increased from UiO-66-0 (pristine UiO-66) (251) to UiO-66-40 (252). The UiO-66-10, UiO-66-20, and UiO-66-30 spectra look similar to the UiO-66-0 spectrum with the characteristic carbonyl stretching frequency between 1392-1386 cm⁻¹ and O—H stretching at 3432-3325 cm⁻¹, which are characteristic features of UiO-66-based MOFs.

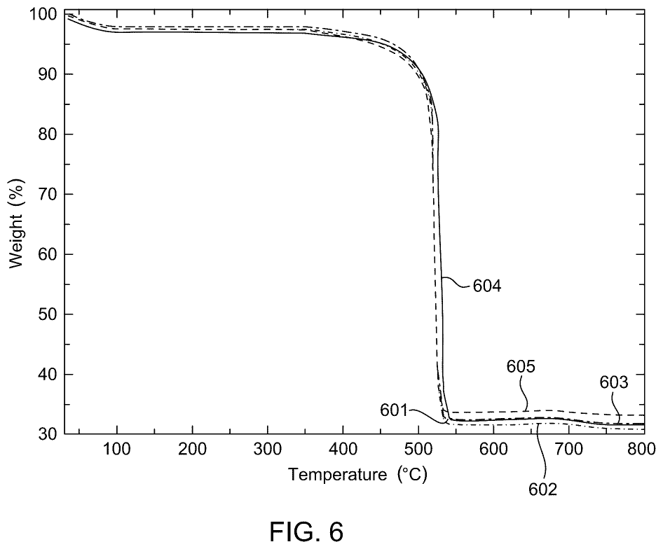

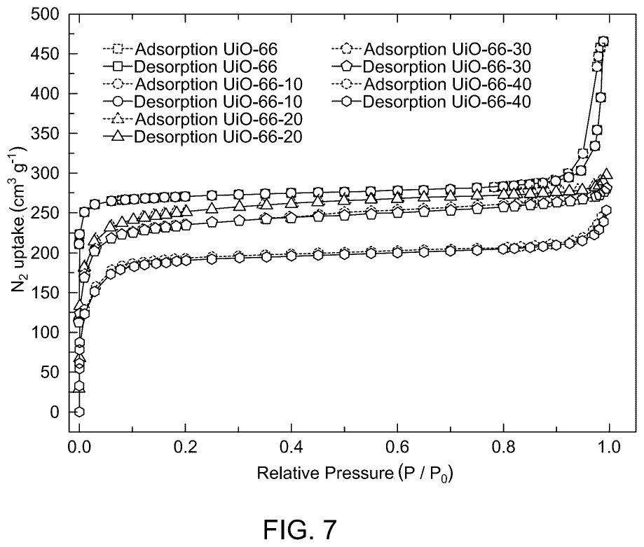

FIG. 6 shows the thermogravimetric analysis (TGA) of pristine UiO-66, UiO-66-0 (601), and UiO-66 with defects, UiO-66-10 (602), UiO-66-20 (603), UiO-66-30 (604), and UiO-66-40 (605). The TGA does not show many changes, which indicates that the UiO-66 with a maximum defect of 40% (UiO-66-40) is thermally stable up to 500° C. This is thought to be due to the strong coordination bond between the hard acid (zirconium) and hard base (carboxylate). FIG. 7 shows the nitrogen (N₂) adsorption isotherms of the pristine UiO-66 and those with varying degrees of linker-modified defects. The nitrogen adsorption isotherm shows a constant decrease in the surface area with the minimum value being UiO-66-40 at 77 K. The decrease in the surface area is attributed to the functionalization of the defects with alkylamine, and thus, the surface area decreases from 1066 m² g⁻¹ to 611 m² g⁻¹ (UiO-66-0>UiO-66-10>UiO-66-20>UiO-66-30>UiO-66-40). The pore distribution increased from 1.3 nm to 1.8 nmwith the increase in the defects.

The reaction of propylene oxide (PO) with CO₂ was chosen as the model reaction to determine the appropriate amount of linker-modified defects for the maximum conversion to cyclic carbonates, and the results are summarized in Table 3. These results indicate that UiO-66 without defects does not lead to any product formation in the presence of CO₂ (1 atm) and heating at 85° C. for 12 hours. There was also no product formation with 10% or 20% defects (UiO-66-10 and UiO-66-20) under similar conditions. As the defects increase from 30% to 40%, the conversion increases from 42% with the maximum conversion of 95% with UiO-66-40. UiO-66-40 was screened for further catalysis experiments under the reaction conditions of 1 bar of CO₂ at 85° C. for 12 hours.

TABLE 3
Cycloaddition reaction of CO₂ with PO in the presence of catalysts
with varying amount of defects^{a}.

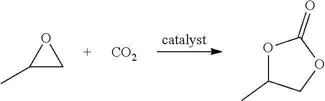

| Entry | Catalyst^{a} | Yield of Cl (%)^{b} |
|---|---|---|
| 1 | UiO-66-0 | No reaction |
| 2 | UiO-66-10 | No reaction |
| 3 | UiO-66-20 | No reaction |
| 4 | UiO-66-30 | 42 |
| 5 | UiO-66-40 | 95 |

^{a}Reaction conditions: PO (6.0 mmol), solvent-free, catalyst (70 mg), CO₂ (1 bar), 12 h, 85° C.;
^{b}Isolated yield calculated with respect to PO

Based on the catalytic performance of UiO-66-40 in the conversion of PO to the PO cyclic carbonate, the efficiency of the catalyst was studied with different aliphatic and aromatic epoxides. As shown in Table 4, UiO-66-40 may efficiently convert all the epoxides to the corresponding cyclic carbonates at 85° C. and for 12 hours. The catalytic conversion rates of propylene oxide, butylene oxide, hexylene oxide, allyl glycidyl ether, and epichlorohydrin to cyclic carbonates are achieved due to the small size of the epoxides which can penetrate the pores of the MOF and can easily access the active sites (acidic and basic sites) of the catalysts. On the other hand, comparatively low conversions were observed for styrene oxide and phenylglycidyl ether due to their bulky structure and steric hindrance toward the active sites of UiO-66-40. The cyclic carbonates yield, and the purities were confirmed by ¹H NMR and ¹³C NMR analyses of the isolated products in all of these catalytic reactions.

TABLE 4
Synthesis of cyclic carbonates from various epoxides

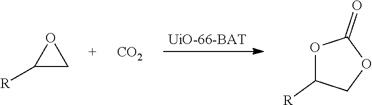

| Entry^{a} | R | Products | Yield (%)^{b} |
|---|---|---|---|
| 1 | —CH₃ | 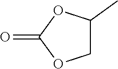 | 95 |
| 2 | —CH₂CH₃ | 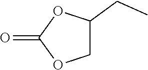 | 92 |
| 3 | —CH₂CH₂CH₂CH₃ | 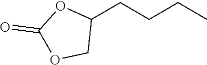 | 93 |
| 4 | 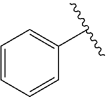 [SMILES: *c1ccccc1] | 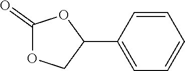 [SMILES: O=C1OCC(c2ccccc2)O1] | 84 |
| 5 | 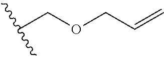 [SMILES: *COCC=C] | 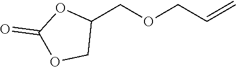 | 91 |
| 6 | —CH₂Cl | 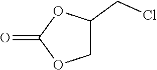 | 93 |
| 7 | 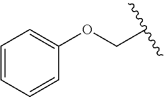 [SMILES: *COc1ccccc1] | 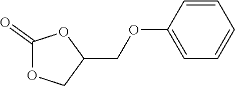 [SMILES: O=C1OCC(COc2ccccc2)O1] | 81 |

^{a}Reaction conditions: Substrate (6 mmol), catalysts (2 mol %), CO₂ (1 bar), 12 hours, 85° C.;
^{b}Yield was determined from ¹H NMR.

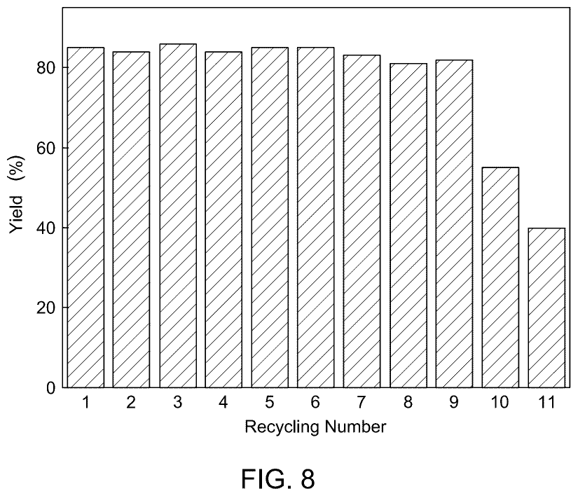

Furthermore, the competence of the catalyst was evaluated by a recyclability test for the conversion of the PO to the corresponding cyclic carbonate. After each cycle of the catalytic reaction, UiO-66-40 was easily recovered by simple centrifugation. The recovered catalyst was thoroughly washed to remove any unreacted PO or product and then dried for re-use in the next cycle. From FIG. 8 it can be seen that UiO-66-40 exhibited good conversion up to nine cycles without the loss of its framework.

The present disclosure describes the modified defect in UiO-66 by the introduction of AMBA as a linker with terephthalic acid. These modified defects not only provide Lewis acid sites for catalytic reactions, but the alkylamine assists in the binding of CO₂ for CO₂ fixation with epoxides in the synthesis of cyclic carbonates. The catalyst was used under ambient pressure, in the absence of co-catalysts, is recyclable for 11 cycles, and does not produce any extra byproducts. The catalyst was applicable for a variety of epoxides with good yield.

Numerous modifications and variations of the present disclosure are possible in light of the above teachings. It is therefore to be understood that within the scope of the appended claims, the invention may be practiced otherwise than as specifically described herein.

1: A method of fixating carbon dioxide with an epoxide to form a substituted cyclic carbonate, comprising:
reacting the epoxide and carbon dioxide in the presence of a zirconium-containing metal-organic framework to form the substituted cyclic carbonate,
wherein the zirconium-containing metal-organic framework is a UiO-66-based metal-organic framework,
wherein the UiO-66-based metal-organic framework comprises an aminomethylbenzoic acid,
wherein the aminomethylbenzoic acid replaces terephthalic acid in the UiO-66-based metal-organic framework.

2: The method of claim 1, wherein the aminomethylbenzoic acid is present in an amount of 5 to 50 mole percent of the total moles of zirconium in the UiO-66-based metal-organic framework.

3: The method of claim 1, wherein a molar ratio of the terephthalic acid to the aminomethylbenzoic acid in the UiO-66-based metal-organic framework is from 1:0.1 to 1:5.

4: The method of claim 1, wherein the aminomethylbenzoic acid is 4-(aminomethyl)benzoic acid.

5: The method of claim 1, wherein the zirconium-containing metal-organic framework has a thermal stability of 450 to 550° C. based on thermogravitimetric analysis.

6: The method of claim 1, wherein the zirconium-containing metal-organic framework is in the form of particles having a BET surface area of 600 to 1000 m²/g.

7: The method of claim 1, wherein the zirconium-containing metal-organic framework is porous and has a pore size distribution of 1.0 to 2.0 nm.

8: The method of claim 1, wherein the epoxide is at least one selected from the group consisting of propylene oxide, butylene oxide, hexylene oxide, allyl glycidyl ether, epichlorohydrin, styrene oxide, and phenyl glycidyl ether.

9: The method of claim 1, wherein the zirconium-containing metal-organic framework is present in an amount of 0.5 to 5 mole percent during the reacting based on a total number of moles of the epoxide, the carbon dioxide, and the zirconium-containing metal-organic framework.

10: The method of claim 1, wherein the reacting occurs at a pressure of 0.5 to 5 bar of carbon dioxide.

11: The method of claim 1, wherein the reacting occurs for a time of 6 to 18 hours.

12: The method of claim 1, wherein the reacting occurs at a temperature of 60 to 120° C.

13: The method of claim 1, wherein the reacting occurs in a liquid phase.

14: The method of claim 1, wherein the percent yield is calculated by proton nuclear magnetic resonance spectroscopy.

15: The method of claim 1, wherein a molar ratio of the terephthalic acid to the aminomethylbenzoic acid in the UiO-66-based metal-organic framework is from 1:0.35 to 1:0.45, the epoxide is propylene oxide, and the percent yield of the cyclic carbonate is 40 to 50 percent with respect to an initial amount of the epoxide.

16: The method of claim 1, wherein a molar ratio of the terephthalic acid to the aminomethylbenzoic acid in the UiO-66-based metal-organic framework is from 1:0.6 to 1:0.8, the epoxide is propylene oxide, and the percent yield of the cyclic carbonate is 93 to 97 percent with respect to an initial amount of the epoxide.

17: The method of claim 1, further comprising making the zirconium-containing metal-organic framework by:
adding a zirconium salt, the terephthalic acid, and the aminomethylbenzoic acid to a solution of N,N-dimethylformamide and acetic acid to form a reaction mixture;
heating the reaction mixture in an autoclave to a temperature of 100 to 150° C. for a time of 20 to 30 hours to form a solid;
washing the solid; and
activating the solid under vacuum at a temperature of 80 to 120° C. for a time of 20 to 30 hours to form the zirconium-containing metal-organic framework.

18: The method of claim 17, wherein a percent yield of the zirconium-containing metal-organic framework is from 70 to 85 weight percent based on the total weight of the zirconium salt.

19: The method of claim 1, comprising:
repeating the reacting in the presence of the zirconium-containing metal-organic framework at least 7 times and up to 9 times,
wherein a yield of a final reacting is at least 80 percent of a yield of an initial reacting based on a cyclic carbonate product yield by proton nuclear magnetic resonance spectroscopy.

20: The method of claim 1, wherein a co-catalyst is not present during the reacting.
